(19) United States
(12) Patent Application Publication
(10) Pub. No.: US 2025/0114143 A1
(43) Pub. Date: Apr. 10, 2025

(54) CATHETER SYSTEM

(71) Applicant: Medtronic Ireland Manufacturing Unlimited Company, Dublin (IE)

(72) Inventors: Somashekharayya Hiremath, Rohnert Park, CA (US); Darion Peterson, Longmont, CO (US); Martha A. Barajas-Torres, Santa Rosa, CA (US)

(21) Appl. No.: 18/695,510

(22) PCT Filed: Oct. 18, 2022
(86) PCT No.: PCT/EP2022/078956

§ 371 Date: Mar. 26, 2024

Related U.S. Application Data
(60) Provisional application No. 63/271,945, filed on Oct. 26, 2021.

(51) Int. Cl. A61B 18/18; A61B 18/00; A61B 90/00

(52) U.S. Cl. CPC A61B 18/1815; A61B 90/08; A61B 2018/00011; A61B 2018/00184; A61B 2018/00577; A61B 2018/1861; A61B 2090/0811

(57) ABSTRACT

A medical system including an inner catheter, an outer jacket, and an expandable section secured to the inner catheter and the outer jacket. The inner catheter supports an energy emitting element configured to emit microwave radiation. The medical system includes an actuator configured to cause the inner catheter to translate relative to the outer jacket to expand the expand or contract the expandable section. The expandable system is configured to radially expand to contact an anatomical lumen wall of a patient to define displacement between the energy emitting element and the anatomical lumen wall. The expandable section may radially expand to substantially center the energy emitting element within the vessel of the patient. The medical system may be used for neuromodulation, such as renal neuromodulation using microwave (MW) energy.

### TECHNICAL FIELD

The present technology is related to neuromodulation catheters. In particular, various examples of the present technology are related to neuromodulation catheters for delivering microwave neuromodulation.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function can be considerably stimulated in disease states characterized by heightened sympathetic tone. For example, reduced renal blood flow and glomerular filtration rate as a result of renal sympathetic efferent stimulation may be a cornerstone of the loss of renal function in cardio-renal syndrome (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter the renal sympathetic mediated sodium and water retention). These pharmacologic strategies, however, can have significant limitations including limited efficacy, compliance issues, side effects, and others.

### SUMMARY

The present technology is directed to devices, systems, and methods for neuromodulation, such as renal neuromodulation using microwave (MW) energy. A medical system (e.g., an MW ablation catheter system) may be configured to deliver microwave energy to tissue around an anatomical lumen (e.g., a renal main artery, accessory renal artery, or branch vessel) in which the catheter is positioned. The medical system may include an inner catheter and an outer jacket, with an expandable section secured to the inner catheter and the outer jacket. The inner catheter supports an energy emitting element configured to emit microwave radiation. The medical system includes an actuator configured to cause relative translation movement between the inner catheter and the outer jacket to expand or contract the expandable section. The expandable section is configured to radially expand to contact a wall of an anatomical lumen when the expandable section is within the anatomical lumen and the medical system causes the relative translational, such that the expandable section defines a displacement between the energy emitting element and the anatomical lumen wall. In examples, the expandable section radially expands to substantially center the energy emitting element within the anatomical lumen. In some instances, this may result in microwave energy delivery in a substantially continuous toroid shape. By delivering microwave energy in such a manner, a substantially continuous circumferential lesion may be formed in tissue, which may reduce a likelihood of renal nerves being left untreated and improve a likelihood of success of the denervation therapy.

In an example, the disclosure describes a medical device for ablation of tissue adjacent an anatomical lumen wall of a patient comprising: an inner catheter; an outer jacket; an expandable section including a first portion secured to the inner catheter and a second portion secured to the outer jacket, an energy emitting element attached to the inner catheter, wherein the energy emitting element is configured to emit microwave energy; and an actuator configured to cause relative translational movement between the inner catheter and the outer jacket to cause the expandable section to radially expand and contract, wherein the expandable section is configured to define a displacement between the energy emitting element and a radially outer surface of the expandable section when the expandable section radially expands to contact the anatomical lumen wall.

In an example, a method comprises radially expanding an expandable section using an actuator to produce relative translational movement between an inner catheter and an outer jacket, wherein the expandable section includes a first portion secured to the inner catheter and a second portion secured to the outer jacket; and emitting microwave energy from an energy emitting element attached to the inner catheter, wherein the expandable section defines a displacement between the energy emitting element and a radially outer surface of the expandable section when the expandable section radially expands to contact an anatomical lumen wall.

Also disclosed herein is a medical system including an inner catheter, an outer jacket, and an expandable section secured to the inner catheter and the outer jacket, wherein the inner catheter supports an energy emitting element configured to emit microwave radiation, wherein the medical system includes an actuator configured to cause the inner catheter to translate relative to the outer jacket to expand the expand or contract the expandable section, wherein the expandable system is configured to radially expand to contact an anatomical lumen wall of a patient to define displacement between the energy emitting element and the anatomical lumen wall, wherein the expandable section may radially expand to substantially center the energy emitting element within the vessel of the patient, and wherein the medical system may be used for neuromodulation, such as renal neuromodulation using microwave (MW) energy.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a schematic illustration of a medical system configured in accordance with some examples of the present disclosure.

FIG. 2 is a schematic illustration of the medical system within an anatomical lumen of a patient.

FIG. 3 is a profile view of the medical system with an expandable section in a delivery configuration.

FIG. 4 is a profile view of the medical system with the expandable section in a deployed configuration.

FIG. 5A is a profile view of the medical system defining a first radial displacement.

FIG. 5B is a profile view of the medical system defining a second radial displacement.

FIG. 5C is a profile view of the medical system defining a third radial displacement.

FIG. 5D is a profile view of the medical system defining a fourth radial displacement.

FIG. 6 is an end view of the medical system with an expandable section in a delivery configuration.

FIG. 7 is an end view of the medical system with an expandable section in a deployed configuration.

FIG. 8 is a profile view of the medical system in a delivery configuration with an energy emitting element between a first expandable portion and a second expandable portion of an expandable section.

FIG. 9 is a profile view of the medical system in a deployed configuration with the energy emitting element between the first expandable portion and the second expandable portion of the expandable section.

FIG. 10A is a profile view of the medical system defining a first radial displacement, with the energy emitting element between the first expandable portion and the second expandable portion of the expandable section.

FIG. 10B is a profile view of the medical system defining a second radial displacement, with the energy emitting element between the first expandable portion and the second expandable portion of the expandable section.

FIG. 10C is a profile view of the medical system defining a third radial displacement, with the energy emitting element between the first expandable portion and the second expandable portion of the expandable section.

FIG. 10D is a profile view of the medical system defining a fourth radial displacement, with the energy emitting element between the first expandable portion and the second expandable portion of the expandable section.

FIG. 11 is a flow diagram of an example technique of using the medical system.

### DETAILED DESCRIPTION

The present technology is directed to devices, systems, and methods for neuromodulation, such as renal neuromodulation, using microwave (MW) energy.

As used herein, the terms “distal” and “proximal” define a position or direction with respect to the treating clinician or clinician's control device (e.g., a actuator). “Distal” or “distally” can refer to a position distant from or in a direction away from the clinician or clinician's control device. “Proximal” and “proximally” can refer to a position near or in a direction toward the clinician or clinician's control device.

Renal neuromodulation, such as renal denervation, may be used to modulate activity of one or more renal nerves and may be used to affect activity of the sympathetic nervous system (SNS). In renal neuromodulation, one or more therapeutic elements may be introduced near renal nerves located between an aorta and a kidney of a patient. In some examples, the one or more therapeutic elements may be carried by or attached to a catheter, and the catheter may be introduced intravascularly, e.g., into a renal artery via a brachial artery, femoral artery, or radial artery approach. In other examples, the one or more therapeutic elements may be introduced extravascularly, e.g., using a laparoscopic technique.

Renal neuromodulation can be accomplished using one or more of a variety of treatment modalities, including electrical stimulation, radio frequency (RF) energy, microwave (MW) energy, ultrasound energy, a chemical agent, or the like. In some examples, a medical system includes a microwave generator configured to generate microwave energy and deliver microwave energy to tissue via an energy emitting element carried by an inner catheter positioned within an anatomical lumen of a body of a patient. For example, the anatomical lumen may be a vessel, such as a vein or artery. In some examples, the anatomical lumen may be a renal artery, such as a main renal artery, a renal vein, an accessory renal artery, a branch vessel, or the like. The microwave energy may heat tissue to which the microwave energy is directed (which tissue includes one or more renal nerves) and modulate the activity of the one or more renal nerves.

The medical system may be configured to transmit microwave energy to tissues of a wall of an anatomical lumen (“anatomical lumen wall”) of a patient when the energy emitting element is within the anatomical lumen. In examples, the energy emitting element is configured to transmit microwave energy to the anatomical lumen wall in a substantially toroidal shape around the energy emitting element. In some examples, the energy emitting element may be configured to transmit microwave energy is a substantially directional manner from the energy emitting element. The transmitted microwave energy may act to increase the kinetic energy of polar molecules (e.g., water) in the anatomical lumen wall, generating heat within the tissues of the anatomical lumen wall (e.g., through dielectric heating). In examples, the medical system is configured to adjust a frequency of the microwave energy emitted to for example, control a depth of microwave penetration in the tissues of the anatomical lumen wall.

The medical system is configured to position the energy emitting element within an anatomical lumen such that the energy emitting element may transmit microwave energy to the anatomical lumen wall. The medical system includes a catheter system including an outer jacket, an inner catheter, and an expandable section. The medical system is configured to cause relative translational movement between the inner catheter and the outer jacket to cause the expandable section to radially expand or contract. For example, the medical system may be configured to translate the inner catheter (proximally or distally) while holding the outer jacket substantially stationary to cause the relative translational movement. The medical system may be configured to translate the outer jacket (proximally or distally) while holding the inner catheter substantially stationary to cause the relative translational movement. The medical system may be configured to translate both the inner catheter (proximally or distally) and the outer jacket (proximally or distally) to cause the relative translational movement. In examples, the outer jacket defines a lumen (“outer jacket lumen”) and the inner catheter is configured to slidably translate within the outer jacket lumen to cause the relative translational movement.

The expandable section is secured to the outer jacket and the inner catheter and configured to radially expand or contract when the medical system causes the relative translational movement between the inner catheter and the outer jacket. In examples, the expandable section includes a first portion secured to the inner catheter and a second portion secured to the outer jacket to cause the radial expansion or contraction when the medical system causes relative translational movement between the inner catheter and the outer jacket. The expandable section may include, for example, a braided structure or another structure configured to radially expand or contract when the medical system causes relative translational movement between the inner catheter and the outer jacket. The expandable section is configured to radially expand to define a displacement between the energy emitting element and a radially outer surface of the expandable section. In examples, the expandable section is configured to radially expand to contact an anatomical lumen wall around a circumference of the anatomical lumen wall when the medical system causes relative translational movement between the inner catheter and the outer jacket, such that the radially expanded expandable section substantially centers at least a portion of the inner catheter (e.g., a portion between a first end and a second end of the expandable section) within an anatomical lumen of the patient.

As discussed herein, when the medical system causes relative translational movement between the inner catheter and the outer jacket, this may mean either the inner catheter, the outer jacket, or both the inner catheter and the outer jacket translate in a manner to alter a position of the inner catheter relative to a position of the outer jacket and/or a position of the outer jacket relative to the inner catheter. In some examples, the medical system is configured to impart motion to the inner catheter to cause the relative translational movement between the inner catheter and the outer jacket. In some examples, the medical system is configured to impart motion to the outer jacket to cause the relative translational movement between the inner catheter and the outer jacket. In some examples, the medical system is configured to impart motion to both the inner catheter and the outer jacket to cause the relative translational movement between the inner catheter and the outer jacket. The medical system may be configured to impart motion to the inner catheter and the outer jacket substantially simultaneously (e.g., substantially at the same time subject to manufacturing tolerances) and/or in a temporally separate manner (e.g., substantially at different times).

The inner catheter may mechanically support the energy emitting element. In examples, the expandable section is configured to define a displacement between the anatomical lumen wall and the energy emitting element when the expandable section radially expands. The expandable section may be configured to define a displacement between the anatomical lumen wall and the energy emitting element substantially circumferentially (e.g., omni-directionally) around the energy emitting element, such that the energy emitting element may radiate microwave energy substantially circumferentially into the anatomical lumen wall to target nerves (e.g., renal nerves) within the anatomical lumen wall. The substantially omni-direction emission may assist in the formation of a substantially continuous circumferential lesion in the tissue of the anatomical lumen wall, which may reduce a likelihood of renal nerves being left untreated and improve a likelihood of success of the denervation therapy.

In examples, the expandable section is configured to assume a delivery configuration defining an initial radial displacement and a deployed configuration defining a deployed radial displacement, with the deployed radial displacement greater than the initial radial displacement. The expandable section may be configured to define a plurality of deployed radial displacements in the deployed configuration. The initial radial displacement and the deployed radial displacement may be displacements substantially perpendicular to a longitudinal axis defined by the inner catheter. The medical system may be configured such that relative translational movement between the inner catheter and the outer jacket causes the expandable section to transition between the delivery configuration and the deployed configuration. In examples, the expandable section is configured to establish the delivery configuration or the deployed configuration based on a compressive state of the expandable section. For example, the expandable section may be configured to establish the delivery configuration when the expandable section is in a relaxed, substantially zero-stress condition, and/or when the expandable section is placed in tension. The expandable section may be configured to establish the deployed configuration when the expandable section is placed in compression (e.g., placed in a compressive state above a threshold compression required to cause the expandable section to establish a deployed configuration). The medical system may be configured such that relative translational movement between the inner catheter and the outer jacket substantially controls the compressive state of the expandable section, such that the translation (e.g., translation of the inner catheter within the outer jacket lumen) causes the expandable section to transition between the delivery configuration and the deployed configuration.

For example, the medical system may be configured to define an axial displacement between a distal end of the outer jacket (“outer jacket distal end”) and a distal end of the inner catheter (“inner catheter distal end”). The medical system may be configured to define a maximum axial displacement between the outer jacket distal end and the inner catheter distal end when the expandable member defines the initial radial displacement (e.g., in the delivery configuration). The medical system may be configured such that a translation of the inner catheter distal end toward the outer jacket distal end and/or a translation of the outer jacket distal end toward the inner jacket distal end (e.g., a decrease in the axial displacement causing a compressive stress) causes the expandable member to transition from the delivery configuration to a deployed configuration. Similarly, the medical system may be configured such that relative translation of the inner catheter distal end away from the outer jacket distal end and/or a translation of the outer jacket distal end away from the inner catheter distal end (e.g., an increase in the axial displacement reducing the compressive stress) causes the expandable member to transition from a deployed configuration to the delivery configuration.

The expandable section may be configured to define a range of deployed radial displacements based on a position of the inner catheter relative to the outer jacket. In examples, a deployed radial displacement defined by the expandable section is proportional to the position of the inner catheter relative to the outer jacket. Hence, the medical system may be configured to cause the expandable section to vary the radial displacement defined by a deployed configuration based on control of the axial displacement between the inner catheter distal end and the outer jacket distal end. In examples, the medical system is configured to establish a plurality of axial displacements between a minimum axial displacement and a maximum axial displacement. The medical system may be configured to vary the axial displacement over a substantially continuous range between the minimum axial displacement and the maximum axial displacement, such that the expandable section may define various radial displacements over a substantially continuous range between the initial radial displacement and a maximum radial displacement (e.g., a maximum radial displacement in a deployed configuration). In examples, the expandable section is configured to define a radial displacement sufficient to cause contact between the expandable section and an anatomical lumen wall of a renal artery of a patient, such as a radial displacement between about 1 mm to about 5 mm.

The expandable section may be configured to allow continued blood flow through the anatomical lumen of a patient when the expandable section establishes a deployed configuration. For example, the expandable section may be configured to provide a plurality of passages defining a flow path for blood flow within the anatomical lumen in the deployed configuration. The plurality of flow passages may facilitate the blood flow for passive blood flow cooling of portions of the anatomical lumen wall as the energy emitting element transmits microwave energy to the anatomical lumen wall, and/or for other reasons. In examples, the expandable section includes a plurality of interweaved braided members configured to separate to define the plurality of passages when the expandable section radially expands. In some examples, the expandable section may be configured to increase a velocity of blood flow at or near the anatomical lumen wall to enhance or accelerate the transfer of heat from the wall to the blood when in a deployed configuration.

The medical system includes an actuator configured to cause the relative translational movement between the inner catheter and the outer jacket. The actuator may be configured to alter the axial displacement defined between the inner catheter distal end and the outer jacket distal end, such that actuator enables control of the radial displacement defined by the expandable section. The actuator may mechanically engage the outer jacket and the inner catheter to cause the relative translational movement between the inner catheter and the outer jacket. In examples, the actuator includes an actuator body and a positioning member configured to move (e.g., be moved by a clinician) relative to the actuator body to cause the relative translational movement between the inner catheter and the outer jacket. For example, the positioning member may include a positioning nut configured to rotate around an axis defined by the actuator body to cause the relative translational movement between the inner catheter and the outer jacket. The actuator may include one or more indicia indicative of the position of the positioning member relative to the actuator body, such as a visible indication, a tactile indication, or another indication sensible by a clinician. The one or more indicia may be configured to enable a clinician to position the positioning member in a specific position relative to the actuator body, such that the clinician may cause the expandable section to establish a particular radial displacement within an anatomical lumen.

In some examples, the actuator is configured to maintain the outer jacket substantially stationary relative to the actuator body and translate the inner catheter relative to the actuator body to cause the relative translational movement between the inner catheter and the outer jacket. In some examples, the actuator is configured to maintain the inner catheter substantially stationary relative to the actuator body and translate the outer jacket relative to the actuator body to cause the relative translational movement between the inner catheter and the outer jacket. In some examples, the actuator is configured to translate the inner catheter relative to the actuator body and translate the outer jacket relative to the actuator body to cause the relative translational movement between the inner catheter and the outer jacket.

In examples, the medical system includes a microwave generator configured to generate microwave energy. The microwave generator may be configured to generate microwaves at various medically acceptable frequencies, such as 915 MHz, 2.45 GHz, and/or 5.1 GHz. The medical system may include a cable (e.g., a transmission line) configured to deliver microwaves from the microwave generator to the energy emitting element. In examples, the medical system includes a control mechanism configured to allow a clinician or other user to initiate, terminate and/or adjust the operation of the microwave generator to, for example, control the delivery of microwave energy from the energy emitting element to an anatomical lumen wall. In some examples, the medical system includes one or more sensors, such as one or more temperature (e.g., thermocouple, thermistor, etc.), impedance, pressure, optical, flow, chemical or other sensors, located proximate to or within the energy emitting element to monitor delivery of microwave energy and/or monitor dielectric heating in the vicinity of the energy emitting element.

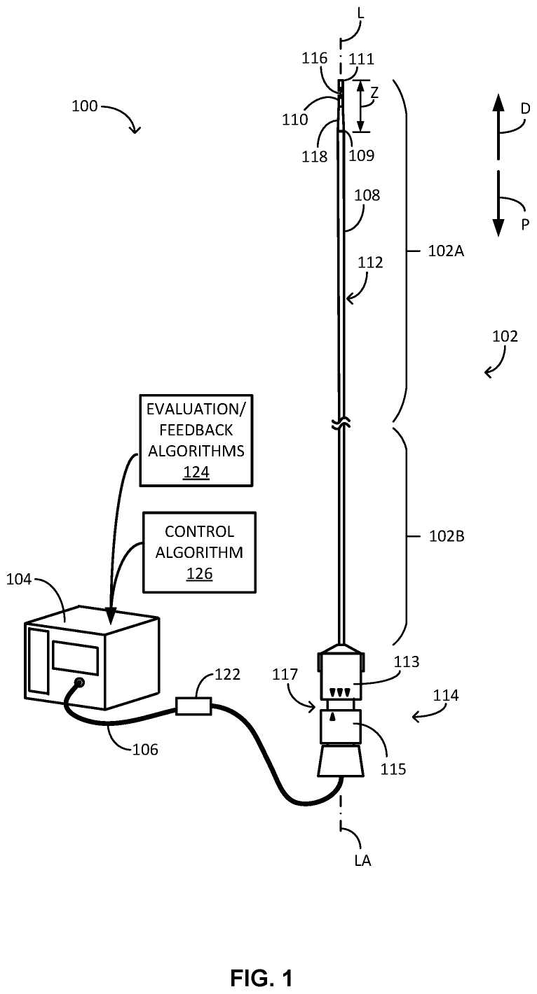

FIG. 1 is a partially schematic perspective view illustrating a medical system 100 configured in accordance with some examples of the present disclosure. Medical system 100 includes a catheter system 102, an microwave generator 104, and a cable 106 extending between catheter 102 and microwave generator 104. Catheter system 102 includes an outer jacket 108 and an inner catheter 110. In examples, outer jacket 108 defines a lumen 112 (“catheter lumen 112”) and inner catheter 110 is configured to slidably translate within catheter lumen 112. In examples, outer jacket 108 defines a distal end 109 (“outer jacket distal end 109”) and inner catheter 110 defines a distal end 111 (“inner catheter distal end 111”). Catheter system 102 may be configured to cause relative translational movement between inner catheter 110 and outer jacket 108 to, for example, alter an axial displacement Z defined between inner catheter distal end 111 and out jacket distal end 109. Catheter system 102 may be configured to impart movement to inner catheter 110, outer jacket 108, and/or inner catheter 110 and outer jacket 108 to cause the relative translational movement between inner catheter 110 and outer jacket 108. Inner catheter 110 mechanically supports energy emitting element 116. Catheter system 102 further includes an actuator 114 configured to cause the relative translational movement between inner catheter 110 and outer jacket 108 (e.g., to alter the axial displacement Z).

In examples, catheter system 102 defines a distal catheter section 102A and a proximal catheter section 102B. Distal catheter section 102A may be configured to locate energy emitting element 116 at a treatment location within or otherwise proximate to an anatomical lumen (e.g., a blood vessel, a duct, an airway, a renal artery, or another naturally occurring anatomical lumen within the human body). In some examples, distal catheter section 102A is configured to locate energy emitting element 116 at an intraluminal (e.g., intravascular) location. Energy emitting element 116 may be configured to provide or support a neuromodulation treatment at the treatment location. In examples, distal catheter section 102A measures 2, 3, 4, 5, 6, or 7 French or another suitable size. Outer jacket 108, catheter lumen 112, and/or inner catheter 110 may extend from distal catheter section 102A through at least some portion of proximal catheter section 102B. In examples, outer jacket 108, catheter lumen 112, and/or inner catheter 110 extend from distal catheter section 102A to at least actuator 114. Distal catheter section 102A and/or proximal catheter section 102B may be configured to flex enroute to a treatment location to position energy emitting element 116 at the treatment location.

Catheter system 102 (e.g., distal catheter section 102A) includes an expandable section 118 configured to radially expand when actuator 114 causes a relative translational movement between inner catheter 110 and outer jacket 108. In examples, expandable section 118 is configured to radially expand in a direction substantially perpendicular to a longitudinal axis L defined by inner catheter 110. Expandable section 118 is secured to outer jacket 108 and inner catheter 110 and configured to radially expand when actuator 114 causes the relative translational movement between inner catheter 110 and outer jacket 108. In examples, expandable section 118 is configured to radially expand when inner catheter distal end 111 moves proximally (e.g., in the proximal direction P) toward outer jacket distal end 109 and/or outer jacket distal end 109 moves distally (e.g., in the distal direction D) toward inner catheter distal end 111 (e.g., when axial displacement Z decreases). When in a deployed configuration, expandable section 118 may be configured to radially contract when inner catheter distal end 111 moves distally (e.g., in the distal direction D) away from outer jacket distal end 109 and/or outer jacket distal end 109 moves proximally (e.g., in the proximal direction P) away from inner catheter distal end 111 (e.g., when axial displacement Z increases).

Longitudinal axis L may be defined by catheter system 102 and extend through catheter system 102 at least from inner catheter distal end 111 to actuator 114. In examples, at least some portion of catheter system 102 (e.g., distal catheter section 102A) is substantially flexible, such that catheter system 102 may flex and/or bend enroute to positioning energy emitting element 116 substantially at a target location within an anatomical lumen of a patient. Hence, although illustrated as substantially linear in FIG. 1, catheter system 102 (or portions thereof) may be configured to assume linear, curved, and/or curvilinear shapes. Correspondingly, longitudinal axis L (and/or portions thereof) defined by catheter system 102 may be linear, curved, and/or curvilinear.

In examples, actuator 114 mechanically engages outer jacket 108 and/or inner catheter 110 to cause the relative translational movement between inner catheter 110 and outer jacket 108 (e.g., to alter the displacement Z). In examples, actuator 114 defines a body 113 (“actuator body 113”) and a positioning member 115 configured to move (e.g., be moved by a clinician) relative to actuator body 113. Actuator 114 may be configured to cause the relative translational movement between inner catheter 110 and outer jacket 108 when positioning member 115 moves relative to actuator body 113. For example, positioning member 115 may include a positioning nut configured to rotate around an axis LA defined by actuator body 113 to cause the relative translational movement between inner catheter 110 and outer jacket 108. In examples, actuator 114 includes one or more indicia 117 indicative of the position of positioning member 115 relative to actuator body 113, such that a clinician may position positioning member 115 in a particular position relative to actuator body 113 to cause expandable member 118 to define a particular radial displacement.

Microwave generator 104 is configured to control, monitor, supply, and/or otherwise support operation of catheter system 102. In other examples, catheter system 102 may be self-contained or otherwise configured for operation independent of microwave generator 104. When present, microwave generator 104 is configured to generate a selected form and/or magnitude of microwave energy for delivery to tissue at a treatment location via energy emitting element 116. For example, microwave generator 104 can be configured to generate microwave energy (e.g., electromagnetic radiation at a frequency greater than about 300 Mhz and less than about 300 Ghz, such as such as 915 MHz, 2.45 GHz, and/or 5.1 GHz). In other examples, microwave generator 104 may be another type of device configured to generate and deliver another suitable type of energy to energy emitting element 116 for delivery to tissue at a treatment location. Microwave generator 104 may include, for example, a cavity magnetron, a klystron, a traveling wave tube, and/or other components suitable for generation of microwave energy. Cable 106 may be configured to electrically transfer microwaves from microwave generator 104 to energy emitting element 116. Cable 106 may include, for example, a coaxial cable or a parallel wire. In examples, cable 106 extends along or within catheter system 102 and/or actuator 114.

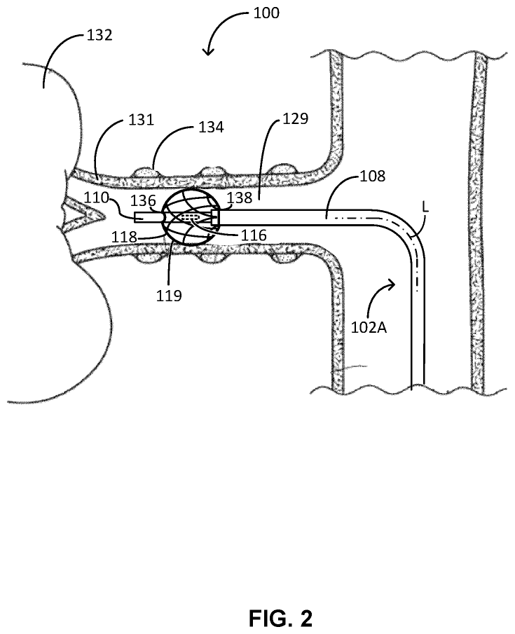

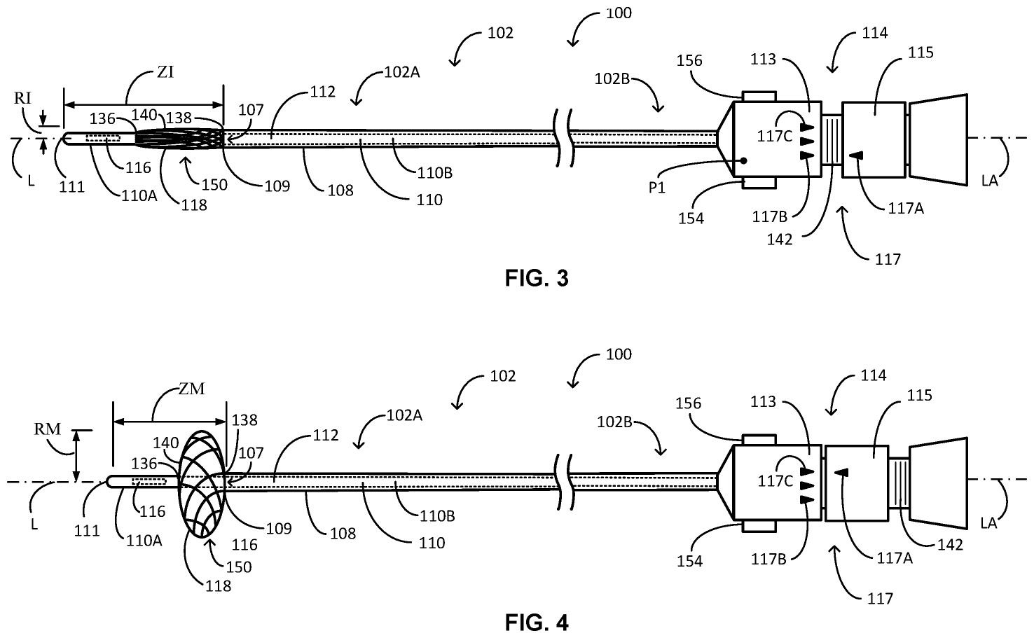

Energy emitting element 116 may be mechanically supported by inner catheter 110. Energy emitting element 116 may be located in catheter system 102 along any location of longitudinal axis L. In examples, inner catheter 110 is configured to position emitting element 116 distal to outer jacket distal end 109 when expandable section 118 is secured to outer jacket 108 and inner catheter 110. In examples, inner catheter 110 is configured to position emitting element 116 distal to expandable section 118 when expandable section 118 is secured to outer jacket 108 and inner catheter 110 (as illustrated in FIGS. 3-5D). In some examples, inner catheter 110 is configured to position energy emitting element 116 such that expandable section 118 substantially extends over (e.g., substantially surrounds) energy emitting element 116 when expandable section 118 is secured to outer jacket 108 and inner catheter 110 (as illustrated in FIG. 2). Energy emitting element 116 may be configured in any manner suitable to receive microwave energy from cable 106 and emit an electromagnetic field (e.g. microwaves) using the microwave energy. Energy emitting element 116 may include, for example, a coaxial antenna and/or other components suitable for emission of the electromagnetic field. In examples, energy emitting element 116 is configured to emit microwaves in a substantially toroidal-shaped, substantially circumferential (e.g., omni-directional) emission around energy emitting element 116. In some examples, energy emitting element 116 may include shielding or other structures to cause a substantially directional (e.g., non-omni-directional) emission of microwaves.

Along cable 106 or at another suitable location within medical system 100, medical system 100 may include a control device 122 configured to initiate, terminate, and/or adjust operation of one or more components of catheter system 102 directly and/or via microwave generator 104. In some examples, microwave generator 104 is configured to execute an automated control algorithm using automated control algorithm circuitry 126 and/or to receive control instructions from an operator. Similarly, in some implementations, microwave generator 104 is configured to provide feedback to an operator before, during, and/or after a treatment procedure via evaluation/feedback algorithm circuitry 124 configured to execute a evaluation/feedback algorithm.

Hence, medical system 100 may be configured such that expandable section 118 may establish a low-profile condition (e.g., in the delivery configuration) for intravascular delivery to an anatomical lumen defined by an anatomical lumen of a patient (e.g., a renal artery). Intraluminal delivery of catheter system 102 (e.g., distal catheter section 102A) may include percutaneously inserting a guidewire (not shown) into an anatomical lumen of a patient and moving at least distal catheter section 102A and energy emitting element 118 along the guide wire until energy emitting element 118 reaches a suitable treatment location. Alternatively, catheter system 102 (e.g., distal catheter section 102A) may be a steerable or non-steerable device configured for use without a guidewire. Additionally, or alternatively, catheter system 102 may be configured for use with another type of guide member, such as a guide catheter or a sheath (not shown), alone, or in addition to a guidewire.

Medical system 100 may be configured such that expandable section 118 may be radially expanded to a deployed configuration within the anatomical lumen when energy emitting element substantially reaches a desired location within the anatomical lumen of the patient. Expandable section 118 may be radially expanded (e.g., by a clinician) using actuator 114, such that expandable section 118 contacts the anatomical lumen wall and substantially centers energy emitting element 116 within the anatomical lumen. With energy emitting element 116 positioned as desired, microwave generator 104 may be utilized to provide microwave energy via cable 105 to energy emitting element 116, such that energy emitting element 116 emits microwaves to induce one or more desired neuromodulating effects on localized regions of the patient. Medical system 100 may be configured such that a clinician may use actuator 114 to cause expandable section 118 to transition from the deployed condition to the delivery configuration for retrieval of catheter system 102 from the anatomical lumen of the patient.

FIG. 2 is a schematic illustration of medical system 100 (e.g., distal catheter section 102A) within an anatomical lumen defined by an anatomical lumen wall of a patient. In the example of FIG. 2, the anatomical lumen is a renal vessel 129, and the anatomical lumen wall is a renal vessel wall 131. Renal vessel 129 is an example of anatomical lumen 128. Renal vessel wall 131 is an example of anatomical lumen wall 130. Expandable section 118 is radially expanded to a deployed configuration to define a displacement between energy emitting element 116 and a radially outer surface 119 of expandable section 118. Radially outer surface 119 may contact renal vessel wall 131to define a displacement between energy emitting element 116 and renal vessel wall 131. In examples, medical system 100 is configured to cause distal catheter section 102A to flex (e.g., using a steering device (not shown)) such that distal catheter section 102A may enter renal vessel 129. In some examples, distal catheter section 102A may be configured to follow a path defined by a guide catheter, a guide wire, or a sheath (not shown) to enter renal vessel 129.

Medical system 100 is configured to substantially maintain expandable section 118 in the low-profile, delivery configuration for intravascular delivery to renal vessel 129. Once energy emitting element 116 is positioned within renal vessel 129, medical system 100 may cause relative translational movement between inner catheter 110 and outer jacket 108 to cause expandable section 118 to radially expand into a deployed configuration. As illustrated in FIG. 2, expandable section 118 may radially expand to contact anatomical lumen wall 130 (e.g., an internal luminal surface of anatomical lumen wall 130). Renal vessel wall 131may substantially center energy emitting element 116 within renal vessel 129 when expandable section 118 radially expands into the deployment configuration. Microwave generator 104 (FIG. 1) may provide microwave energy to tissue of the patient by radiation from energy emitting element 116 to induce one or more desired neuromodulation effects on localized regions of anatomical lumen 128 and regions adjacent to anatomical lumen 128. For example, when anatomical lumen wall 130 defines a renal artery, energy emitting element 116 may induce one or more desired neuromodulating effects to a portion of Renal Plexus (RP) 134 lying within or adjacent to the adventitia of a renal artery. Medical system 100 is configured to cause a radial contraction of expandable section 118 from the deployed configuration to the delivery configuration for retrieval of distal catheter section 102A from anatomical lumen 128.

Energy emitting element 116 may be located in catheter system 102 along any location of longitudinal axis L. In examples, inner catheter 110 is configured to position emitting element 116 distal to outer jacket distal end 109 when expandable section 118 is secured to outer jacket 108 and inner catheter 110. In some examples, as illustrated in FIG. 2, inner catheter 110 is configured to position energy emitting element 116 such that expandable section 118 substantially extends over (e.g., substantially surrounds) energy emitting element 116 when expandable section 118 is secured to outer jacket 108 and inner catheter 110. In some examples, as illustrated in FIGS. 3-5D, inner catheter 110 is configured to position emitting element 116 distal to expandable section 118 when expandable section 118 is secured to outer jacket 108 and inner catheter 110.

Neuromodulation effects can include thermal ablation, non-ablative thermal alteration, coagulation or damage (e.g., via sustained heating and/or dielectric heating), or electromagnetic neuromodulation. The microwave energy emitted by energy emitting element 118 may cause dielectric heating which raises the temperature of target neural fibers above a certain threshold to achieve non-ablative thermal alteration, or above a higher temperature to achieve ablative thermal alteration. For example, the target temperature can be above body temperature (e.g., approximately 37° Celsius (C) but less than about 45° C. for non-ablative thermal alteration, or the target temperature can be about 45° C. or higher for ablative thermal alteration. Desired non-thermal neuromodulation effects may include altering the electrical signals transmitted in a nerve.

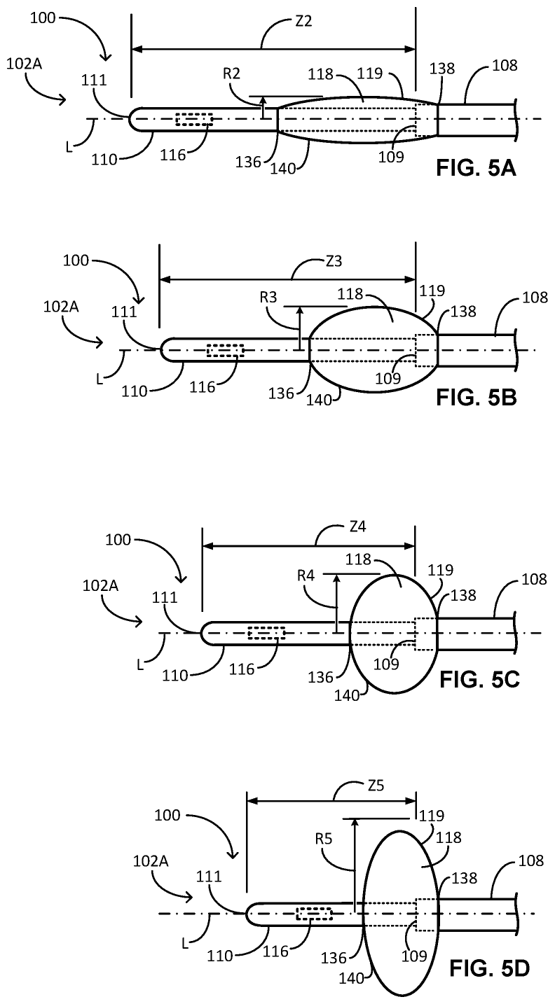

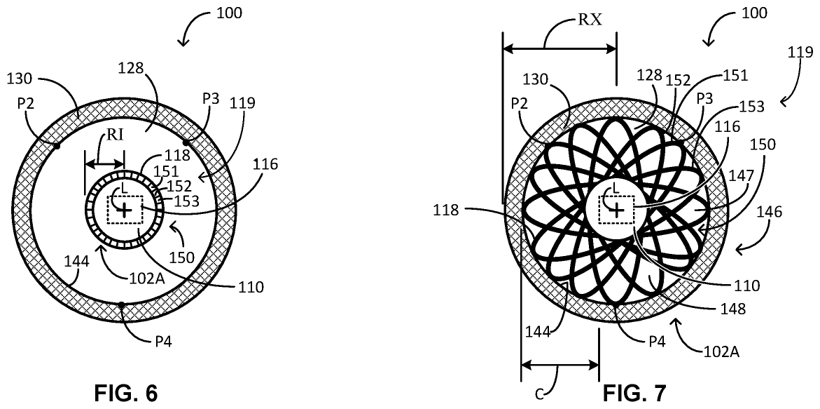

FIG. 3 illustrates a schematic profile view of medical system 100 with expandable section 118 in a delivery configuration defining a minimum radial displacement RI. FIG. 4 illustrates a schematic profile view of medical system 100 with expandable section 118 in a deployed configuration defining a maximum radial displacement RM. FIG. 5A, FIG. 5B, FIG. 5C, and FIG. 5D illustrate schematic profile views of medical system 100 with expandable section 118 in deployed configurations defining radial displacements R3, R4, R5, and R6 respectively, with radial displacements R3, R4, R5, and R6 greater than the minimum radial displacement RI and less than the maximum radial displacement RM. FIG. 6 is a schematic end view of medical system 100 with expandable section 118 in a delivery configuration and positioned within anatomical lumen 128 defined by anatomical lumen wall 130. FIG. 7 is a schematic end view of medical system 100 with expandable section 118 in a deployed configuration and positioned within anatomical lumen 128 defined by anatomical lumen wall 130.

Actuator 114 is configured to cause relative translational movement between inner catheter 110 and outer jacket 108 to cause expandable section 118 to radially expand or contract. For example, actuator 114 may be configured to cause the relative translational movement to alter the axial displacement between outer jacket distal end 109 and inner catheter distal end 111. In examples, inner catheter 110 is configured to translate (e.g., slidably translatable) relative to outer jacket 108 within catheter lumen 112 defined by outer jacket 108. As used herein, when inner catheter 110 translates relative to outer jacket 108 (e.g., within catheter lumen 112), this may mean inner catheter 110 moves relative to a fixed point P1 while outer jacket 108 remains substantially stationary relative to the fixed point P1, or may mean outer jacket 108 moves relative to the fixed point P1 while inner catheter 110 remains substantially stationary relative to the fixed point P1, or may mean inner catheter 110 and outer jacket 108 move relative to the fixed point P1. In examples, outer jacket distal end 109 defines an opening 107 to catheter lumen 112 (“catheter lumen opening 107”). Catheter lumen 112 may extend from catheter lumen opening 107 and into at least proximal catheter section 102B of catheter system 102. In examples, catheter lumen 112 extends from catheter lumen opening 107 to at least actuator 114.

Inner catheter 110 may be configured to extend through catheter lumen 112 such that inner catheter distal end 111 is distal to catheter lumen opening 107. In examples, catheter lumen opening 107 is configured (e.g., sized) to allow at least a portion of inner catheter 110 to pass therethrough when inner catheter 110 is positioned within catheter lumen 112. In examples, inner catheter 110 defines a distal portion 110A (“inner catheter distal portion 110A) configured to be distal to catheter lumen opening 107 when inner catheter 110 is positioned within catheter lumen 112. Inner catheter 110 may define a proximal portion 110B (“inner catheter proximal portion 110B”) configured to be proximal to catheter lumen opening 107 when inner catheter 110 is positioned within catheter lumen 112. In examples, inner catheter proximal portion 110B extends from catheter lumen opening 107 and into at least proximal catheter section 102B. Inner catheter proximal portion 110B may extends from catheter lumen opening 107 to at least actuator 114.

Expandable section 118 may be secured to outer jacket 108 and inner catheter 110 such that expandable section 118 radially expands or contracts when actuator 114 causes relative translational movement between inner catheter 110 and outer jacket 108 (e.g., when actuator 114 causes inner catheter 110 to translate relative to outer jacket 108 within catheter lumen 112). In examples, expandable section 118 includes a first portion 136 (“first expandable portion 136”) secured to inner catheter 110. Expandable section 118 may include a second portion 138 (“second expandable portion 138”) secured to outer jacket 108. In examples, first expandable portion 136 is configured to remain substantially stationary with respect to inner catheter 110 and/or second expandable portion 138 is configured to remain substantially stationary with respect to outer jacket 108, such that when actuator 114 causes relative translational movement between inner catheter 110 and outer jacket 108, the relative translational movement causes similar relative movement between first expandable portion 136 and second expandable portion 138. In examples, the relative translational movement between inner catheter 110 and outer jacket 108 alters a compressive state of expandable section 118. For example, expandable section 118 may be configured such that when relative translational movement between inner catheter 110 and outer jacket 108 decreases a distance between first expandable portion 136 and second expandable portion 138, expandable section 118 experiences an increase in compressive forces between first expandable portion 136 and second expandable portion 138. Expandable section 118 may be configured such that when relative translational movement between inner catheter 110 and outer jacket 108 increases the distance between first expandable portion 136 and second expandable portion 138, expandable section 118 experiences a decrease in compressive forces between first expandable portion 136 and second expandable portion 138.

In examples, expandable section 118 includes a medial portion 140 (“expandable medial portion 140”) between first expandable portion 136 and second expandable portion 138. Expandable section 118 may be configured to establish a delivery configuration or the deployed configuration based on the compressive state of expandable medial portion 140. For example, expandable section 118 may be configured to establish a minimum radial displacement RI (FIG. 3) when expandable medial section 140 is in a relaxed, substantially zero-stress condition, and/or when expandable medial section 140 is placed in tension. Expandable section 118 may be configured to establish a deployed radial displacement such as a maximum radial displacement RM (FIG. 4), when expandable medial section 140 is placed in compression. In examples, medial portion 140 is configured to substantially buckle when a compressive load is applied between first expandable portion 136 and second expandable portion 138. The buckling may cause expandable medial section 140 to define the deployed radial displacement. Medial portion 140 may be a substantially elastic section, such that placing medial portion 140 in a zero-stress or tensile state from the buckled condition causes medial portion 140 to establish the minimum radial displacement RI.

Expandable section 118 may be configured to define a radial displacement based on an axial displacement established and/or maintained by catheter system 102 between inner catheter distal end 111 and outer jacket distal end 109. In examples, expandable section 118 is configured to define a minimum radial displacement RI when catheter system 102 establishes a maximum axial displacement ZI between inner catheter distal end 111 and outer jacket distal end 109 (FIG. 3). Expandable section 118 may be configured to define a maximum radial displacement RM when catheter system 102 establishes a minimum axial displacement ZM between inner catheter distal end 111 and outer jacket distal end 109 (FIG. 4). The minimum radial displacement RI and/or the maximum radial displacement RM may define respective distances substantially perpendicular to longitudinal axis L. The maximum axial displacement ZI and/or the minimum axial displacement ZM may define respective distances substantially along longitudinal axis L. As will be discussed below, catheter system 102 may be configured to define one or more medial axial displacements defining distances between those defined by the maximum axial displacement ZI and the minimum axial displacement ZM, such that expandable section 118 may define one or more medial radial displacements defining distances between those defined by the minimum radial displacement RI and the maximum radial displacement RM. In examples, catheter 102 may be configured to establish any medial axial displacement, to cause expandable section 118 to define any medial radial displacement. Actuator 114 may be configured to cause relative translational movement between inner catheter 110 and outer jacket 108 to cause catheter system 102 to define the maximum axial displacement ZI, the minimum axial displacement ZM, and/or a medial axial displacement defining a distance between those defined by the maximum axial displacement ZI and the minimum axial displacement ZM.

For example, FIG. 5A illustrates catheter system 102 defining an axial displacement Z2 causing expandable section 118 (e.g., expandable medial portion 140) to radially expand to define a radial displacement R2. Axial displacement Z2 may be less than axial displacement ZI (FIG. 3) and radial displacement R2 may be greater than radial displacement RI (FIG. 3). FIG. 5B illustrates catheter system 102 defining an axial displacement Z3 causing expandable section 118 (e.g., expandable medial portion 140) to radially expand to define a radial displacement R3. Axial displacement Z3 may be less than axial displacement Z2 and radial displacement R3 may be greater than radial displacement R2. FIG. 5C illustrates catheter system 102 defining an axial displacement Z4 causing expandable section 118 (e.g., expandable medial portion 140) to radially expand to define a radial displacement R4. Axial displacement Z4 may be less than axial displacement Z3 and radial displacement R4 may be greater than radial displacement R3. FIG. 5D illustrates catheter system 102 defining an axial displacement Z5 causing expandable section 118 (e.g., expandable medial portion 140) to radially expand to define a radial displacement R5. Axial displacement Z5 may be less than axial displacement Z4 and greater than axial displacement ZM (FIG. 4), and radial displacement R5 may be greater than radial displacement R4 and less than radial displacement RM (FIG. 4). For clarity, expandable section 118 is illustrated as a transparent component defined by radially outer surface 119 of expandable section 118.

As discussed, expandable section 118 is configured to define a radial displacement to cause contact between expandable section 118 and anatomical lumen wall 130 when expandable section 118 radially expands from the delivery configuration to a deployed configuration. As an example, FIG. 6 schematically illustrates an end view of distal catheter section 102A within anatomical lumen 128 with expandable section 118 defining the minimum radial displacement RI (e.g., corresponding to maximum axial displacement ZI (FIG. 3)). Inner catheter 110 mechanically supports energy emitting element 116 within anatomical lumen 128. FIG. 7 schematically illustrates an end view of distal catheter section 102A with expandable section 118 having expanded to define a radial displacement RX, where the radial displacement RX causes contact between anatomical lumen wall 130 and expandable section 118. The radial displacement RX may be one of radial displacement R2, radial displacement R3, radial displacement R4, radial displacement R5, maximum radial displacement RD, or another radial displacement in a range bounded by minimum radial displacement RI and maximum radial displacement RD. Actuator 114 may be configured to cause relative translational motion between inner catheter 110 and outer jacket 108 to cause expandable section 118 to expand, such that expandable section 118 transitions from defining the radial displacement RI to defining the radial displacement RX. Similarly, actuator 114 may be configured to cause relative translational motion between inner catheter 110 and outer jacket 108 to cause expandable section 118 to contract, such that expandable section 118 transitions from defining the radial displacement RX to defining the radial displacement RI.

Expandable section 118 may be configured to expand to contact anatomical lumen wall 130 at one or more points on a perimeter (e.g., a circumference) defined by anatomical lumen wall 130 and surrounding longitudinal axis L when distal catheter section 102A is positioned within anatomical lumen 128 (e.g., by a clinician). For example, expandable section 118 may be configured to contact anatomical lumen wall 130 at one or more points such as point P2, point P3, and/or point P4 on a perimeter defined on inner surface 144 of anatomical lumen wall 130. In examples, expandable section 118 is configured to contact inner surface 144 to limit movement of expandable section 118 and/or energy emitting element 116 within anatomical lumen 128. For example, expandable section 118 may be configured to contact point P2, point P3, and/or point P4 such that, when expandable section 118 exerts a force on one of point P2, point P3, and/or point P4, inner surface 144 exerts an oppositely oriented reaction force through point P2, point P3, and/or point P4 to limit movement of expandable section 118 and/or energy emitting element 116 within anatomical lumen 128.

In examples, expandable section 118 is configured to define a displacement C between energy emitting element 116 and radially outer surface 119 when expandable section 118 radially expands to contact anatomical lumen wall 130. Expandable section 118 may be configured to limit movement of energy emitting element 116 relative to anatomical lumen wall 130 to, for example, substantially maintain the displacement C when expandable section 118 is in a deployed configuration. In some examples, expandable section 118 is configured to substantially center energy emitting element 116 within anatomical lumen 128 when expandable section 118 is in a deployed configuration, as shown in, for example, FIG. 7.

Expandable section 118 may be configured to allow continued blood flow through anatomical lumen 128 when expandable section 118 is radially expanded to establish a deployed configuration. Expandable section 118 may be configured to provide a plurality of passages 146 defining a flow path for blood flow within anatomical lumen 128 when expandable section 118 radially expands, such as passage 147 and passage 148. In some examples, expandable section 118 may be configured to increase a velocity of blood flow at or near anatomical lumen wall 130 (e.g., inner surface 144) to enhance or accelerate the transfer of heat from anatomical lumen wall 130 to the blood flowing through anatomical lumen 128.

In examples, expandable section 118 includes a plurality of braided members 150, such as member 151, member 152, and member 153. One or more of member 151, member 152, and/or member 153 may define radially outer surface 119. The plurality of braided members 150 may be configured to separate to define the plurality of passages 146 when expandable section 118 radially expands. In examples, the plurality of braided members 150 are interweaved, such that a radial expansion of a braided member is constrained unless one or more other braided members likewise expand, and/or radial contraction of the braided member is constrained unless one or more other braided members likewise contract. In examples, a braided member (e.g., member 151) is in slidable contact with and interwoven with at least two other braided members (e.g., member 152 and member 153), and the braided member is configured to separate from the at least two other braided members to define the plurality of passages 146 when expandable section 118 radially expands.

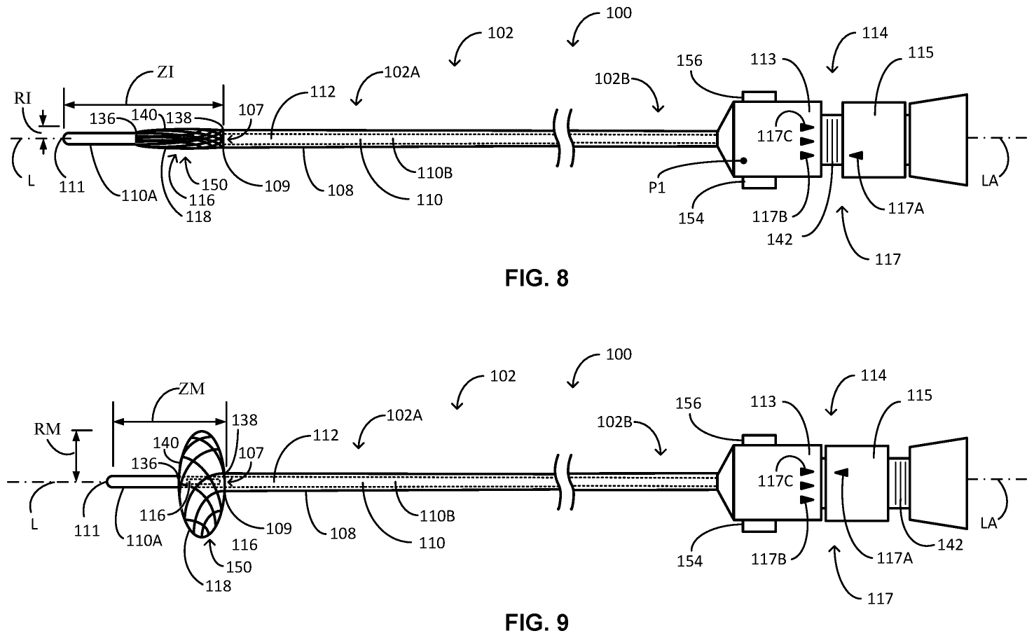

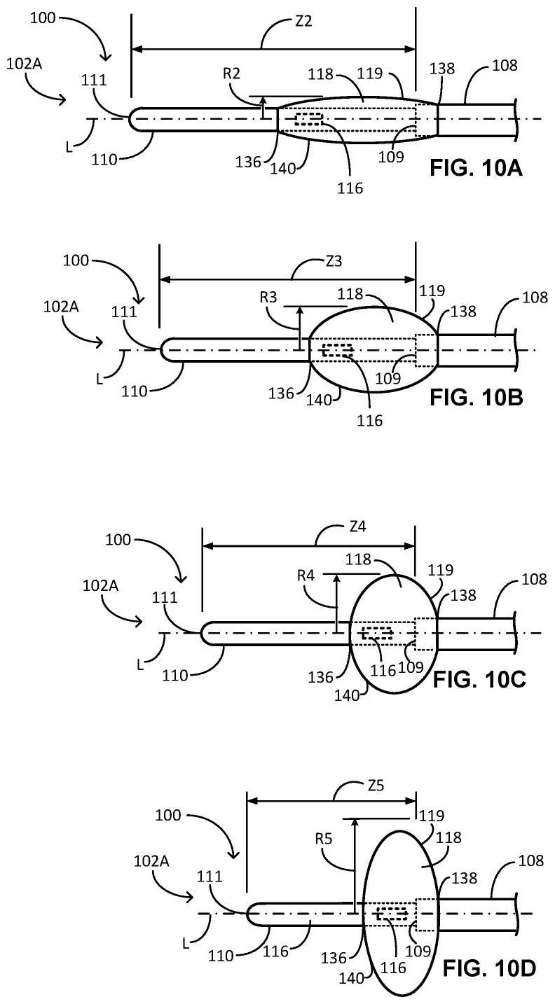

Inner catheter 110 may mechanically support energy emitting element 116 at any position on inner catheter 110. In examples, inner catheter 110 mechanically supports energy emitting element 116 such that energy emitting element 116 is distal to outer jacket distal end 109. In examples, inner catheter 110 mechanically supports energy emitting element 116 such that energy emitting element 116 is distal to inner catheter distal end 111. In some examples, as illustrated at FIG. 3, FIG. 4, and FIG. 5A-5D, inner catheter 110 mechanically supports energy emitting element 116 such that energy emitting element 116 is distal to first expandable portion 136 of expandable element 118 when medical system 100 is in a delivery configuration and/or deployed configuration. In some examples, as illustrated at FIG. 2, FIG. 8, FIG. 9, and FIGS. 10A-10D, inner catheter 110 mechanically supports energy emitting element 116 such that energy emitting element 116 is proximal to first expandable portion 136 and distal to second expandable portion 138 (e.g., between first expandable portion 136 and second expandable portion 138 and/or within an internal volume defined by expandable element 118) when medical system 100 is in a delivery configuration and/or deployed configuration. In some examples, inner catheter 110 mechanically supports energy emitting element 116 such that energy emitting element 116 is distal to first expandable portion 136 and proximal to second expandable portion 138 when medical system 100 is in a delivery configuration and/or deployed configuration. Inner catheter 110 may be configured to mechanically support energy emitting element 116 such that energy emitting element 116 is substantially stationary (e.g., axially and/or radially) relative to inner catheter 110.

For example, FIG. 8 illustrates a schematic profile view of medical system 100 with expandable section 118 in a delivery configuration defining the minimum radial displacement RI, with inner catheter 110 mechanically supporting energy emitting element 116 such that energy emitting element 116 is proximal to first expandable portion 136 and distal to second expandable portion 138. FIG. 9 illustrates a schematic profile view of medical system 100 with expandable section 118 in a deployed configuration defining the maximum radial displacement RM, with inner catheter 110 mechanically supporting energy emitting element 116 such that energy emitting element 116 is proximal to first expandable portion 136 and distal to second expandable portion 138. FIG. 10A, FIG. 10B, FIG. 10C, and FIG. 10D illustrate schematic profile views of medical system 100 with expandable section 118 in deployed configurations defining radial displacements R3, R4, R5, and R6 respectively, with inner catheter 110 mechanically supporting energy emitting element 116 such that energy emitting element 116 is proximal to first expandable portion 136 and distal to second expandable portion 138. FIG. 8, FIG. 9, FIG. 10A, FIG. 10B, FIG. 10C, and FIG. 10D depict the same and/or similar operations and/or configurations of medical system 100 as FIG. 3, FIG. 4, FIG. 5A, FIG. 5B, FIG. 5C, and FIG. 5D respectively, with the exception of inner catheter 110 mechanically supporting energy emitting element 116 such that energy emitting element 116 is proximal to first expandable portion 136 and distal to second expandable portion 138.

Expandable section 118 may comprise a metallic material, a ceramic material, a plastic, a polymeric material such as polytetrafluoroethylene (PFTE) or another polymer, or another suitable material. In examples (e.g., when energy emitting element 116 is mechanically supported between first expandable portion 136 and second expandable portion 138), expandable section 118 may comprise a material substantially transparent to microwave radiation (e.g., a material with a low dielectric constant). In examples, expandable section 118 comprises a thermoplastic such as polyether ether ketone.

Actuator 114 (FIG. 3, FIG. 4) may be configured to cause relative translational movement between inner catheter 110 and outer jacket 108 to cause catheter system 102 to define axial displacements in addition to maximum axial displacement ZI, axial displacement Z2, axial displacement Z3, axial displacement Z4, axial displacement Z5, and maximum axial displacement ZM (FIGS. 3-5D). Actuator 114 may be configured to cause expandable medial portion 140 to define radial displacements in addition to minimum radial displacement RI, radial displacement R2, radial displacement R3, radial displacement R4, radial displacement R5, and maximum radial displacement RM (FIGS. 3-5D). In examples, actuator 114 is configured to cause catheter system 102 to define axial displacements in a substantially continuous range bounded by ZI and ZM. Actuator 114 may be configured to cause catheter system 102 to define radial displacements in a substantially continuous range bounded by RI and RM. Hence, actuator 114 may be utilized by a clinician to substantially tailor the radial expansion of expandable section 118 to cause contact between expandable section 118 and anatomical lumen wall 130 (FIG. 2) over a range of potential dimensions (e.g., diameters) defined by anatomical lumen 128.

The axial displacement defined by catheter system 102 may be defined as a distance along longitudinal axis L between inner catheter distal end 111 and outer jacket distal end 109. In examples, the axial displacement is a distance along longitudinal axis L between a first line intersecting inner catheter distal end 111 and longitudinal axis L and a second line intersecting outer jacket distal end 109 and longitudinal axis L, where the first line and the second line are substantially perpendicular to longitudinal axis L. Correspondingly, the axial displacement may define a displacement occurring over a path defined by longitudinal axis L. Hence, the path (and/or portions thereof) defining the axial displacement defined by catheter system 102 may be linear, curved, and/or curvilinear. The radial displacement defined by expandable section 118 (e.g., expandable medial section 140) may be defined as a distance defined between expandable section 118 and longitudinal axis L. In examples, the radial displacement is a maximum distance defined between expandable section 118 and longitudinal axis L, such as a distance defined between radially outer surface 119 and longitudinal axis L. In some examples, the radial displacement is substantially perpendicular to longitudinal axis L. As used herein, “substantially perpendicular” may refer to defining an angle of greater than 75 degrees, greater than 85 degrees, greater than 89 degrees, or defining an angle of 90 degrees.

Actuator 114 may be configured to indicate the axial displacement defined by catheter system 102 using indica 117. For example, positioning of positioning member 115 in a first specific position relative to actuator body 113 may cause catheter system 102 to establish the maximum axial displacement ZI corresponding to the delivery configuration. Indicia 117 may be configured to indicate (e.g., to a clinician) when positioning member 115 is in the first specific position relative to actuator body 113, such that indicia 117 indicate when expandable section 118 is in the delivery configuration. Indicia 117 may be configured to provide a visible indication (e.g., using a visible marker), a tactile indication (e.g., using a detent), or another indication sensible by the clinician. In examples, actuator 114 is configured such that a relative position (e.g., a substantial alignment) between an indicia 117A (e.g., on positioning member 115) and an indicia 117B (e.g., on actuator body 113) indicates that catheter system 102 has established and/or is maintaining the initial axial displacement ZI, such that expandable section 118 has assumed and/or is remaining in the delivery configuration.

Similarly, positioning of positioning member 115 in a second specific position relative to actuator body 113 may cause catheter system 102 to establish a particular axial displacement such as axial displacement Z2, Z3, Z4, Z5, ZM, or another axial displacement between the maximum axial displacement ZI and the minimum axial displacement ZM, such that medical system 100 is in a deployed configuration. Indicia 117 may be configured to indicate (e.g., to a clinician) when positioning member 115 is in the second specific position relative to actuator body 113. For example, actuator 114 may be configured such that a relative position (e.g., a substantial alignment) between indicia 117A (e.g., on positioning member 115) and indicia 117C (e.g., on actuator body 113) indicates that catheter system 102 has established and/or is maintaining the particular axial displacement (such as the minimum axial displacement ZM). In examples, actuator 114 is configured such that a spatial relationship observable by a clinician (e.g., a visible, tactile, or other indication observable by a clinician) between a first indicia on actuator body 113 (e.g, indicia 117B and or indicia 117C) and a second indicia on positioning member 115 (e.g., indicia 117A) is indicative of an axial displacement defined by catheter system 102.

Actuator 114 may be configured to define a specific axial displacement when positioning member 115 establishes a specific position relative to actuator body 113 (e.g., relative to fixed point P1 on actuator body 113). In examples, movement of positioning member 115 relative to actuator body 113 causes relative translational movement between inner catheter 110 and outer jacket 108. In some examples, positioning member 115 is configured to exert a force on inner catheter 110 to cause translation of inner catheter 110 when positioning member 115 moves relative to actuator body 113. In some examples, positioning member 115 is configured to exert a force on outer jacket 108 to cause translation of outer jacket 108 when positioning member 115 moves relative to actuator body 113. In some examples, positioning member 115 is configured to exert a force on inner catheter 110 and outer jacket 108 to cause translation of inner catheter 110 (e.g., in a first direction) and outer jacket 108 (e.g., in a second direction substantially opposite the first direction) when positioning member 115 moves relative to actuator body 113. In examples, actuator 114 is configured to hold one of inner catheter 110 or outer jacket 108 substantially stationary relative to actuator body 113 when positioning member 115 causes a translation of the other of inner catheter 110 or outer jacket 108. Positioning member 115 may be configured to alter the displacement Z (FIG. 1) defined by catheter system 102 when positioning member 115 causes translation of inner catheter 110 and/or outer jacket 108. As discussed, medical system 100 may be configured such the displacement Z defined by catheter system 102 controls the radial displacement defined by expandable section 118. Hence, positioning member 115 may be configured to control the radial displacement defined by expandable section 118 based on the position of positioning member 115 relative to actuator body 113.

In some examples, positioning member 115 is configured to rotate around an axis LA defined by actuator body 113 to cause the translation of inner catheter 110 and/or outer jacket 108. For example, positioning member 115 may be configured to rotate around the axis LA in a first direction to translate inner catheter 110 and/or outer jacket 108 to increase the displacement Z. Positioning member 115 may be configured to rotate around the axis LA in a second direction opposite the first direction to translate inner catheter 110 and/or outer jacket 108 to decrease the displacement Z. In some examples, actuator body 113 defines a thread set 142 and positioning member 115 is configured to threadably engage thread set 142. Thread set 142 may be configured to cause positioning member 115 displace relative to actuator body 113 when positioning member 115 rotates around axis LA. Positioning member 115 may be configured such that the displacement caused by thread set 142 causes positioning member 115 to exert a force on inner catheter 110 and/or outer jacket 108.

In some examples, actuator 114 may be configured to prevent and/or limit an overexpansion of expansion element 118. Actuator 114 may include a release mechanism (not shown) configured to decrease a radial expansion of expandable section 118 when a force exerted on expandable section 118 (e.g. a reaction force exerted by anatomical lumen wall 130 through point P2, point P3, and/or P4 (FIG. 7) exceeds a threshold. Expandable section 118 may be configured to exert a force on the release mechanism substantially proportional to the reaction force exerted by anatomical lumen wall 130 on expandable section 118. In examples, the release mechanism is configured to cause relative translational movement between inner catheter 110 and outer jacket 108 to decrease a radial displacement of expandable section 118 when the force exerted on expandable section 118 exerts the threshold. The release mechanism may include, for example, a spring or other stored energy device configured to cause relative translational movement between inner catheter 110 and outer jacket 108. In examples, the spring comprises a constant force spring such as, for example a torsion spring (e.g., a spiral wound torsion spring).

Medical system 100 may be configured to provide a fluid flow to, for example, provide cooling to energy emitting element 118 and/or other portions of medical system 100. In examples, inner catheter 110 is configured to a flow path for a cooling fluid, wherein the flow path is configured to establish heat transfer between one or more portions of medical system 100 (e.g., energy emitting element 116) and a fluid flowing through the flow path. In examples, actuator 114 (e.g., actuator body 113) defines a flow inlet 154 configured to provide the cooling fluid to the flow path defined by internal catheter 110. Actuator 114 (e.g., actuator body 113) may define a flow outlet 156 configured to receive the cooling fluid from the flow path defined by internal catheter 110. Medical system 100 may include a temperature sensor, such as a thermistor or thermocouple, positioned within or in the vicinity of the flow path (e.g., in the vicinity of energy emitting element 116) to monitor a temperature. Temperature data collected with the temperature sensor may be utilized in a feedback loop to control and/or alter delivery of the microwave field and/or the cooling fluid in response to the temperature (e.g., to maintain the temperature within a desired range). For example, the temperature data collected with the temperature sensor may be utilized by evaluation/feedback algorithm circuitry 124 and/or control algorithm circuitry 126 to control and/or alter delivery of the microwave field and/or the cooling fluid.

Microwave generator 104, evaluation/feedback algorithm circuitry 124 and/or control algorithm circuitry 126, as well as other control circuitry described herein, can comprise any suitable arrangement of hardware, software, firmware, or any combination thereof, to perform the techniques attributed to medical system 100 herein. For example, microwave generator 104, evaluation/feedback algorithm circuitry 124 and/or control algorithm circuitry 126, may include any one or more microprocessors, digital signal processors (DSPs), application specific integrated circuits (ASICs), field programmable gate arrays (FPGAs), or any other equivalent integrated or discrete logic circuitry, as well as any combinations of such components.

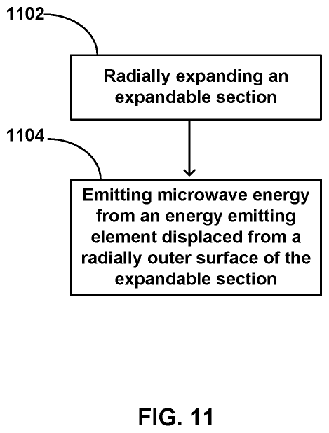

An example technique for positioning a medical system 100 within an anatomical lumen 128 is illustrated in FIG. 11. Although the technique is described mainly with reference to medical system 100 of FIGS. 1-10D, the technique may be applied to other medical systems in other examples.

The technique includes radially expanding an expandable section 118 of a medical system 100 using an actuator 114 to produce relative translational movement between an inner catheter 110 and an outer jacket 108 (1102). Actuator 114 may cause relative translational movement between a first expandable portion 136 secured to inner catheter 110 and a second expandable portion 138 secured to outer jacket 108 when actuator 114 produces the relative translational movement. Expandable section 118 may radially expand to contact an anatomical lumen wall 130 when actuator 114 causes the relative translational movement between inner catheter 110 and outer jacket 108.

Expandable section 118 may radially expand when a distal catheter section 102A is positioned within an anatomical lumen 128 defined by anatomical lumen wall 130. Actuator 114 may mechanically engage a proximal catheter portion 102B positioned outside anatomical lumen 128 to cause the relative translational movement between inner catheter 110 and outer jacket 108. In examples, actuator 114 causes relative translational movement between inner catheter 110 and outer jacket 108 when a positioning member 115 of actuator 114 moves relative to an actuator body 113 of actuator 114. Actuator 114 may cause the relative translational movement by translating inner catheter 110 relative to actuator body 113, translating outer jacket 108 relative to actuator body 113, and/or translating inner catheter 110 and outer jacket 108 relative to actuator body 113. In examples, inner catheter 110 moves relative to outer jacket 108 within a catheter lumen 112 defined by outer jacket 108 when actuator 114 causes relative translational movement between inner catheter 110 and outer jacket 108.

Actuator 114 may alter an axial displacement defined between an inner catheter distal end 111 of inner catheter 110 and an outer jacket distal end 109 of outer jacket 108 to cause the relative translational movement between inner catheter 110 and outer jacket 108. Actuator 114 may control a radial displacement of expandable section 118 using the axial displacement. In examples, expandable section 118 defines a minimum radial displacement RI when actuator 114 establishes and/or maintains a maximum axial displacement ZI to place medical system 100 in a delivery configuration. In examples, expandable section 118 defines a radial displacement greater than the minimum radial displacement RI when actuator 114 establishes and/or maintains an axial displacement less than the maximum axial displacement ZI to place medical system 100 in a deployed configuration. In examples, expandable section 118 defines a maximum radial displacement RM when actuator 114 establishes and/or maintains a minimum axial displacement ZM. The technique may include positioning medical system 100 within anatomical lumen 128 with medical system 100 in the delivery configuration, expanding expandable section 118 to place medical system 100 in a deployed configuration within anatomical lumen 128, and contracting expandable section 118 to return medical system 100 to the delivery condition to retrieve medical system 100 from anatomical lumen 128.

Actuator 114 may alter the axial displacement defined between an inner catheter distal end 111 and outer jacket distal end 109 using a positioning member 115. Positioning member 115 may cause the axial displacement to alter when positioning member 115 moves relative to actuator body 113. In examples, positioning member 115 rotates around an axis LA defined by actuator body 113 to moves relative to actuator body 113. Actuator 114 may control the radial expansion and contraction of expandable section 118 using the motion of positioning member 115 relative to actuator body 113.

The technique includes emitting microwave energy from an energy emitting element 116 attached to inner catheter 110 when expandable element 118 is radially expanded (1104). Expandable section 118 defines a displacement C between energy emitting element 118 and a radially outer surface 119 of expandable section 118 when expandable section 118 radially expands. In examples, radially outer surface 119 contacts an inner surface 144 of anatomical lumen wall 130 when expandable section 118 radially expands. Energy emitting element 116 may emit microwave energy to tissues in or in proximity to anatomical lumen wall 130. In examples, energy emitting element 116 emits microwave energy substantially circumferentially (e.g., omni-directionally) around longitudinal axis L. In examples, energy emitting element 116 emits microwave energy substantially directionally (e.g., non-omni-directionally) relative to energy emitting element 116.

Energy emitting element 116 may emit microwave energy from a position distal to outer sheath distal end 109. In examples, energy emitting element 116 emits microwave energy from a position distal to inner catheter distal end 111. In some examples, energy emitting element 116 emits microwave energy from a position proximal to first expandable portion 136 and distal to second expandable portion 138. In some examples, energy emitting element 116 substantially spans a displacement between first expandable portion 136 and second expandable portion 138 when energy emitting element 116 emits microwave energy.

In examples, expandable section 118 defines a plurality of passages 146 when expandable section 118 radially expands. The plurality of passages 146 may establish fluid communication through expandable section 118 when expandable section 118 radially expands. A plurality of braided members 150 may defines the plurality of passages 146 when expandable section 118 radially expands. In examples, at least one braided member 151 in slidable contact with and weaved through a braided member 152 and a braided member 153 separates from braided member 152 and 153 to define plurality of passages 146 when expandable section 118 radially expands.

Catheters configured in accordance with at least some embodiments of the present technology can be well suited (e.g., with respect to sizing, flexibility, operational characteristics, and/or other attributes) for performing renal neuromodulation in human patients. Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically-induced, thermally-induced, or induced in another suitable manner or combination of manners at one or more suitable treatment locations during a treatment procedure. The treatment location can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery.

Renal neuromodulation can include a microwave-based and/or electrode-based treatment modality alone or in combination with another treatment modality. Microwave-based treatment can include delivering microwaves and/or another form of energy to tissue at or near a treatment location to stimulate and/or heat the tissue in a manner that modulates neural function. For example, sufficiently stimulating and/or heating at least a portion of a sympathetic renal nerve can slow or potentially block conduction of neural signals to produce a prolonged or permanent reduction in renal sympathetic activity.

Heating effects of microwave-based treatment can include ablation and/or non-ablative alteration or damage (e.g., via sustained heating and/or resistive heating). For example, a treatment procedure can include raising the temperature of target neural fibers to a target temperature above a first threshold to achieve non-ablative alteration, or above a second, higher threshold to achieve ablation. The target temperature can be higher than about body temperature (e.g., about 37° Celsius (C)) but less than about 45° C. for non-ablative alteration, and the target temperature can be higher than about 45° C. for ablation. Heating tissue to a temperature between about body temperature and about 45° C. can induce non-ablative alteration, for example, via moderate heating of target neural fibers or of luminal structures that perfuse the target neural fibers. In cases where luminal structures are affected, the target neural fibers can be denied perfusion resulting in necrosis of the neural tissue. Heating tissue to a target temperature higher than about 45° C. (e.g., higher than about 60° C.) can induce ablation, for example, via substantial heating of target neural fibers or of luminal structures that perfuse the target fibers. In some patients, it can be desirable to heat tissue to temperatures that are sufficient to ablate the target neural fibers or the luminal structures, but that are less than about 90° C. (e.g., less than about 85° C., less than about 80° C., or less than about 75° C.).

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure. For example, while particular features of the neuromodulation catheters were described as being part of a single device, in other examples, these features can be included on one or more separate devices that can be positioned adjacent to and/or used in tandem with the neuromodulation catheters to perform similar functions to those described herein.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within the single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique, or any combination thereof.

Various examples of the disclosure have been described. Any combination of the described systems, operations, or functions is contemplated. These and other examples are within the scope of the following claims.

Further disclosed herein is the subject-matter of the following clauses:

1. A medical device for ablation of tissue adjacent an anatomical lumen wall of a patient comprising:
an inner catheter;
an outer jacket;
an expandable section including a first portion secured to the inner catheter and a second portion secured to the outer jacket,
an energy emitting element attached to the inner catheter, wherein the energy emitting element is configured to emit microwave energy; and
an actuator configured to cause relative translational movement between the inner catheter and the outer jacket to cause the expandable section to radially expand and contract,
wherein the expandable section is configured to define a displacement between the energy emitting element and a radially outer surface of the expandable section when the expandable section radially expands to contact the anatomical lumen wall.
   2. The medical device of clause 1, wherein the inner catheter defines a longitudinal axis, and wherein the expandable section is configured to radially expand in a direction substantially perpendicular to the longitudinal axis.
   3. The medical device of clause 1 or clause 2, wherein the energy emitting element is configured to remain substantially radially stationary with respect to the inner catheter when the actuator causes relative translational movement between the inner catheter and the outer jacket.
   4. The medical device of any of clauses 1-3, wherein the outer jacket defines a lumen, and wherein the inner catheter is configured to slidably translate within the lumen when the actuator causes relative translational movement between the inner catheter and the outer jacket.
   5. The medical device of clause 4, wherein the outer sheath is configured to flex to define a curvature, and wherein the inner catheter is configured to flex within the lumen when the outer sheath defines the curvature.
   6. The medical device of any of clauses 1-5, wherein the inner catheter defines a longitudinal axis, and wherein the actuator is configured to cause the expandable section to define a radial displacement from the longitudinal axis.
   7. The medical device of clause 6, wherein the expandable section is configured to maintain the substantially constant radius from the longitudinal axis when the outer sheath flexes to define a curvature.
   8. The medical device of any of clauses 1-7, wherein expandable section is configured to define a plurality of passages establishing fluid communication through the expandable section when the expandable section is radially expanded.
   9. The medical device of clause 8, wherein the expandable section comprises a plurality of braided members, wherein each braided member is in slidable contact with and weaved through at least two other braided members, and wherein the each braided member is configured to separate from the at least two other braided members to define the plurality of passages when the expandable section is radially expanded.
   10. The medical device of any of clauses 1-9, wherein the radial expansion and contraction of the expandable section is proportional to a distance the inner catheter translates relative to the outer jacket.
   11. The medical device of clause 10, wherein the actuator is configured to control the translated distance.
   12. The medical device of any of clauses 1-11, wherein the inner catheter is configured to position the energy emitting element between the distal end of the expanding member and the proximal end of the expanding member.
   13. The medical device of any of clauses 1-11, wherein the inner catheter is configured to position the energy emitting element distal to the distal end of the expanding member.
   14. The medical device of any of clauses 1-13, wherein the actuator mechanically engages a proximal end of the inner catheter and a proximal end of the outer jacket to cause the relative translational movement between the inner catheter and the outer jacket.
   15. The medical device of any of clauses 1-14, wherein the actuator defines an actuator body and a positioning member configured to move relative to the actuator body, and wherein the actuator is configured to cause the relative translational movement between the inner catheter and the outer jacket when the positioning member moves relative to the actuator body.
   16. The medical device of clause 15, wherein the outer sheath is configured to be substantially stationary relative to the actuator body when the positioning member moves relative to the actuator body.
   17. The medical device of clause 15 or clause 16, wherein the positioning member comprises a positioning nut configured to rotate around an axis defined by the actuator body.
   18. The medical device of any of clauses 15-17, wherein the positioning member is configured to impart a force on the inner catheter to cause the relative translational movement between the inner catheter and the outer jacket when the positioning member moves relative to the actuator body.
   19. The medical device of any of clauses 15-17, wherein the positioning member is configured to impart a force on the outer sheath to cause the relative translational movement between the inner catheter and the outer jacket when the positioning member moves relative to the actuator body.
   20. The medical device of any of clauses 15-19, wherein the radial expansion and contraction of the expandable section is proportional to the motion of the positioning member relative to the actuator body.
   21. The medical device of clause 20, wherein the actuator is configured to indicate one or more positions of the positioning member relative to the actuator body, wherein each of the one or more positions correspond to a specific displacement between the expandable section and a longitudinal axis defined by the inner catheter.
   22. The medical device of any of clauses 1-21, wherein the actuator includes a release mechanism, wherein the expandable section is configured to exert a force on the release mechanism when the expandable section contacts the anatomical lumen wall of the vasculature, and wherein the release mechanism is configured to decrease the radial expansion of the expandable section when the force exerted exceeds a threshold.
   23. The medical device of clause 22, wherein the release mechanism includes a spring configured to cause a translation of the inner catheter relative to the outer sheath when the force exerted exceeds the threshold.
   24. The medical device of clause 23, wherein the spring comprises a constant force spring.
   25. The medical device of clause 23 of clause 24, wherein the spring comprises a torsion spring.
   26. The medical device of any of clauses 1-25, wherein the inner catheter defines a longitudinal axis, and wherein the energy emitting element is configured to emit the microwave energy in a substantially radial direction from the longitudinal axis and circumferentially around the axis when the expandable section is radially expanded.
   27. The medical device of any of clauses 1-26, wherein the inner catheter defines a fluid intake and a fluid discharge, wherein the inner catheter is configured to receive a cooling fluid through the fluid intake, transfer heat from the energy emitting element to the cooling fluid to generate a heated cooling fluid, and discharge the heated cooling fluid from the fluid discharge.
   28. The medical device of any of clauses 1-27, wherein the energy emitting element is configured to emit microwave energy to ablate the tissue adjacent the anatomical wall.
   29. The medical device of any of clauses 1-28, wherein the expandable section is configured to define a displacement between the energy emitting element and the anatomical lumen wall when the expandable section radially expands to contact the anatomical lumen wall.
   30. A method, comprising:
radially expanding an expandable section using an actuator to produce relative translational movement between an inner catheter and an outer jacket, wherein the expandable section includes a first portion secured to the inner catheter and a second portion secured to the outer jacket; and
emitting microwave energy from an energy emitting element attached to the inner catheter, wherein the expandable section defines a displacement between the energy emitting element and a radially outer surface of the expandable section when the expandable section radially expands to contact an anatomical lumen wall.
   31. The method of clause 30, further comprising radially expanding the expandable member in a direction substantially perpendicular to a longitudinal axis defined by the inner catheter.
   32. The method of clause 30 or 31, further comprising translating the energy emitting element relative to the outer jacket when the actuator produces relative translational movement between the inner catheter and the outer jacket.
   33. The method of any of clauses 30-32, further comprising slidably translating the inner catheter within a lumen defined by the outer jacket when the actuator produces relative translational movement between the inner catheter and the outer jacket.
   34. The method of clause 33, further comprising flexing the outer sheath to define a curvature, and flexing the inner catheter within the lumen when the outer sheath defines the curvature.
   35. The method of any of clauses 30-34, further comprising maintaining a substantially constant radius of the expandable section from a longitudinal axis defined by the inner catheter when the outer sheath flexes to define a curvature.
   36. The method of any of clauses 30-35, further comprising defining a plurality of passages with the expandable section to establish fluid communication through the expandable section when the expandable section is radially expanded.
   37. The method of clause 36, further comprising defining the plurality of passages using a plurality of braided members comprising the expandable section, wherein each braided member is in slidable contact with and weaved through at least two other braided members, and wherein the each braided member is configured to separate from the at least two other braided members to define the plurality of passages when the expandable section is radially expanded.
   38. The method of any of clauses 30-37, further comprising controlling the radial expansion and contraction of the expandable section using the actuator to control an axial displacement between a distal end of the inner catheter and a distal end of the outer jacket.
   39. The method of any of clauses 28-36, further comprising positioning the energy emitting element between the distal end of the expanding member and the proximal end of the expanding member when the actuator produces relative translational movement between the inner catheter and the outer jacket.
   40. The method of any of clauses 30-39, further comprising positioning the energy emitting element distal to the distal end of the expanding member when the actuator produces relative translational movement between the inner catheter and the outer jacket.
   41. The method of any of clauses 30-40, further comprising mechanically engaging the actuator and a proximal end of the inner catheter and mechanically engaging the actuator and a proximal end of the outer jacket to produce the relative translational movement between the inner catheter and the outer jacket.
   42. The method of any of clauses 30-41, further comprising moving a positioning member of the actuator relative to an actuator body defined by the actuator to cause the relative translational movement between the inner catheter and the outer jacket.
   43. The method of clause 42, further comprising maintaining the outer sheath substantially stationary relative to the actuator body when the positioning member moves relative to the actuator body.
   44. The method of clause 42 or clause 43, further comprising rotating a positioning nut around an axis defined by the actuator body to cause the relative translational movement between the inner catheter and the outer jacket.
   45. The method of any of clauses 42-44, further comprising imparting a force to the inner catheter to cause the relative translational movement between the inner catheter and the outer jacket when the positioning member moves relative to the actuator body.
   46. The method of any of clauses 42-45, further comprising imparting a force to the outer jacket to cause the relative translational movement between the inner catheter and the outer jacket when the positioning member moves relative to the actuator body.
   47. The method of any of clauses 42-46, further comprising controlling the radial expansion and contraction of the expandable section using the motion of the positioning member relative to the actuator body.
   48. The method of any of clauses 42-47, further comprising establishing a specific displacement between the expandable section and a longitudinal axis defined by the inner catheter by establishing the positioning member in a specific position relative to the actuator body.
   49. The method of any of clauses 42-48, further comprising exerting a force on the release mechanism using the expandable section, wherein the release mechanism is configured to decrease the radial expansion of the expandable section when the force exerted exceeds a threshold.
   50. The method of any of clauses 30-49, further comprising emitting the microwave energy from the energy emitting element in a substantially radial direction from and circumferentially around a longitudinal axis defined by the inner catheter when the expandable section is radially expanded.
   51. The method of any of clauses 30-50, further comprising:
receiving a cooling fluid through a fluid intake defined by the inner catheter, the outer jacket, or the actuator;
transferring heat from the energy emitting element to the cooling fluid; and
discharging the heated cooling fluid from a fluid discharge defined by the inner catheter, the outer jacket, or the actuator.
   52. The method of any of clauses 30-51, wherein the expandable section defines a minimum radial displacement when the inner catheter and the outer catheter define a maximum axial displacement, and wherein radially expanding the expandable section comprises establishing a medial axial displacement using the relative translational movement, wherein the medial axial displacement is less than the maximum axial displacement.
   53. The method of clause 52, further comprising defining a medial radial displacement of the expandable section when the relative translational movement establishes the medial axial displacement, wherein the medial radial displacement is greater than the minimum radial displacement.
   54. The method of any of clauses 30-51, wherein the expandable section defines a maximum radial displacement when the inner catheter and the outer catheter define a minimum axial displacement, and further comprising contracting the expandable section by establishing a medial axial displacement using the relative translational movement, wherein the medial axial displacement is greater than the minimum axial displacement.
   55. The method of clause 54, further comprising defining a medial radial displacement of the expandable section when the relative translational movement establishes the medial axial displacement, wherein the medial radial displacement is less than the maximum radial displacement.

1. A medical device for ablation of tissue adjacent an anatomical lumen wall of a patient comprising:
an inner catheter;
an outer jacket;
an expandable section including a first portion secured to the inner catheter and a second portion secured to the outer jacket;
an energy emitting element attached to the inner catheter, wherein the energy emitting element is configured to emit microwave energy; and
an actuator configured to cause relative translational movement between the inner catheter and the outer jacket to cause the expandable section to radially expand or contract,
wherein the expandable section is configured to define a displacement between the energy emitting element and a radially outer surface of the expandable section when the expandable section radially expands to contact the anatomical lumen wall.

2. The medical device of claim 1, wherein the inner catheter defines a longitudinal axis, and wherein the expandable section is configured to radially expand in a direction substantially perpendicular to the longitudinal axis when the actuator causes relative translational movement between the inner catheter and the outer jacket.

3. The medical device of claim 1, wherein the outer jacket defines a lumen, and wherein the inner catheter is configured to slidably translate within the lumen when the actuator causes relative translational movement between the inner catheter and the outer jacket.

4. The medical device of claim 1, wherein the inner catheter defines a longitudinal axis, and wherein the actuator is configured to cause the expandable section to define a radial displacement from the longitudinal axis.

5. The medical device of claim 1, wherein the expandable section is configured to define a plurality of passages establishing fluid communication through the expandable section at least when the expandable section is radially expanded.

6. The medical device of claim 1, wherein the radial expansion and contraction of the expandable section is proportional to a distance the inner catheter translates relative to the outer jacket, and wherein the actuator is configured to control the translated distance.

7. The medical device of claim 1, wherein the inner catheter is configured to at least one of position the energy emitting element between the distal end of the expanding member and the proximal end of the expanding member, or position the energy emitting element distal to the distal end of the expanding member.

8. The medical device of claim 1, wherein the actuator mechanically engages a proximal end of the inner catheter and a proximal end of the outer jacket to cause the relative translational movement between the inner catheter and the outer jacket, wherein the actuator defines an actuator body and a positioning member configured to move relative to the actuator body, and wherein the actuator is configured to cause the relative translational movement between the inner catheter and the outer jacket when the positioning member moves relative to the actuator body.

9. The medical device of claim 8, wherein the actuator is configured to indicate one or more positions of the positioning member relative to the actuator body, and wherein each of the one or more positions correspond to a specific displacement between the expandable section and a longitudinal axis defined by the inner catheter.

10. The medical device of claim 1, wherein the actuator includes a release mechanism, wherein the expandable section is configured to exert a force on the release mechanism when the expandable section contacts the anatomical lumen wall of the vasculature, and wherein the release mechanism is configured to decrease the radial expansion of the expandable section when the force exerted exceeds a threshold.

11. The medical device of claim 1, wherein the inner catheter defines a longitudinal axis, and wherein the energy emitting element is configured to emit the microwave energy in a substantially radial direction from the longitudinal axis and circumferentially around the axis when the expandable section is radially expanded.

12. A method, comprising:
radially expanding an expandable section using an actuator, wherein the actuator is configured to produce relative translational movement between an inner catheter and an outer jacket, wherein the expandable section includes a first portion secured to the inner catheter and a second portion secured to the outer jacket; and
emitting microwave energy from an energy emitting element attached to the inner catheter, wherein the expandable section defines a displacement between the energy emitting element and a radially outer surface of the expandable section when the expandable section radially expands to contact an anatomical lumen wall.

13. The method of claim 12, wherein radially expanding the expandable section comprises radially expanding the expandable section in a direction substantially perpendicular to a longitudinal axis defined by the inner catheter, and using the actuator to produce relative translational movement between the inner catheter and the outer jacket and translate the energy emitting element relative to the outer jacket.

14. The method of claim 12, wherein the expandable member defines a plurality of passages that establish fluid communication through the expandable section when the expandable section is radially expanded.

15. The method of claim 12, further comprising:
mechanically engaging the actuator and a proximal end of the inner catheter;
mechanically engaging the actuator and a proximal end of the outer jacket; and
moving a positioning member of the actuator relative to an actuator body defined by the actuator to cause a relative translational movement between the inner catheter and the outer jacket.

16. The method of claim 15, further comprising imparting a force to at least one of the inner catheter or the outer jacket to cause the relative translational movement between the inner catheter and the outer jacket when the positioning member moves relative to the actuator body.

17. The method of claim 15, further comprising controlling the radial expansion and contraction of the expandable section using the motion of the positioning member relative to the actuator body.

18. The method of claim 12, further comprising:
causing, using a fluid intake defined by the inner catheter, the outer jacket, or the actuator, the fluid intake to receive a cooling fluid;
transferring heat from the energy emitting element to the cooling fluid; and
discharging the heated cooling fluid from a fluid discharge defined by the inner catheter, the outer jacket, or the actuator.

19. The method of claim 12, wherein the expandable section defines a maximum radial displacement when the inner catheter and the outer catheter define a minimum axial displacement, the method further comprising contracting the expandable section by establishing a medial axial displacement using the relative translational movement, wherein the medial axial displacement is greater than the minimum axial displacement.

20. The medical device of claim 1, wherein the energy emitting element is configured to remain substantially radially stationary with respect to the inner catheter when the actuator causes relative translational movement between the inner catheter and the outer jacket.

21. The medical device of claim 1, wherein the inner catheter defines a fluid intake and a fluid discharge, wherein the inner catheter is configured to receive a cooling fluid through the fluid intake, and cause a heat transfer from the energy emitting element to the cooling fluid to generate a heated cooling fluid, and discharge the heated cooling fluid from the fluid discharge.

22. The medical device of claim 5, wherein the expandable section comprises a plurality of braided members, wherein each braided member is in slidable contact with and weaved through at least two other braided members, and wherein each braided member is configured to separate from the at least two other braided members to define the plurality of passages when the expandable section is radially expanded.

23. The medical device of claim 8, wherein the radial expansion and contraction of the expandable section is proportional to the motion of the positioning member relative to the actuator body.

24. A medical device comprising:
an inner catheter;
an outer jacket;
an expandable section including a first portion secured to the inner catheter and a second portion secured to the outer jacket;
an energy emitting element attached to the inner catheter; and
an actuator configured to cause relative translational movement between the inner catheter and the outer jacket to cause the expandable section to radially expand or contract,
wherein the expandable section is configured to define a displacement between the energy emitting element and a radially outer surface of the expandable section when the expandable section radially expands to contact the anatomical lumen wall, and
wherein the expandable section is configured to define a plurality of passages establishing fluid communication through the expandable section at least when the expandable section radially expands.

25. The medical device of claim 24, wherein the actuator includes a release mechanism, wherein the expandable section is configured to exert a force on the release mechanism when the expandable section contacts the anatomical lumen wall of the vasculature, and wherein the release mechanism is configured to decrease the radial expansion of the expandable section when the force exerted exceeds a threshold.
